(19) United States
(12) Patent Application Publication
(10) Pub. No.: US 2007/0265487 A1
(43) Pub. Date: Nov. 15, 2007

(54) Applicators for use in positioning implants for use in brachytherapy and other radiation therapy

(75) Inventors: Gary A. Lamoureux, Woodbury, CT (US); Warren W. Johnston, Thomaston, CT (US)

Correspondence Address: FLIESLER MEYER LLP, 650 CALIFORNIA STREET, 14TH FLOOR, SAN FRANCISCO, CA, 94108, US

(73) Assignee: WORLDWIDE MEDICAL TECHNOLOGIES LLC, Oxford, CT (US)

(21) Appl. No.: 11/592,865

(22) Filed: Nov. 3, 2006

Related U.S. Application Data
(60) Provisional application No. 60/798,973, filed on May 9, 2006.
(60) Provisional application No. 60/836,160, filed on Aug. 8, 2006.

(51) Int. Cl. A61N 5/00; A61M 36/00

(52) U.S. Cl. 600/7; 600/3

(57) ABSTRACT

In one embodiment an applicator includes a needle receiver adapted to be removably mateable with a needle positioned at the surgical site, a housing adapted to receive a push rod, and a clip for receiving one or more implants, the clip having a plurality of ports adapted to bridge the needle receiver and the housing so that the needle receiver can receive the push rod, wherein the clip can be rotatably repositioned so that the plurality of ports can selectively bridge the needle receiver and the housing. This abstract is not intended to be a complete description of the invention.

### CLAIM OF PRIORITY

This application claims priority to the following United States Provisional Applications, which are incorporated herein by reference.

U.S. Provisional Application Ser. No. 60/798,973, entitled “After-loader for Positioning Implants for Needle Delivery in Brachytherapy and Other Radiation Therapy,” filed May 9, 2006.

U.S. Provisional Application Ser. No. 60/836,160, entitled “After-loader for Positioning Implants for Needle Delivery in Brachytherapy and Other Radiation Therapy,” filed Aug. 8, 2006.

### FIELD OF THE INVENTION

This invention relates to radiotherapy. More particularly, it relates to applicators for positioning implants e.g., for use in brachytherapy.

### BACKGROUND

Brachytherapy is a general term covering medical treatment which involves placement of radioactive sources near a diseased tissue and can involve the temporary or permanent implantation or insertion of radioactive sources into the body of a patient. The radioactive sources are located in proximity to the area of the body which is being treated. A high dose of radiation can thereby be delivered to the treatment site with relatively low doses of radiation to surrounding or intervening healthy tissue. Exemplary radioactive sources include radioactive seeds, radioactive rods and radioactive coils.

Brachytherapy has been used or proposed for use in the treatment of a variety of conditions, including arthritis and cancer. Exemplary cancers that can be treated using brachytherapy include breast, brain, liver and ovarian cancer and especially prostate cancer in men. For a specific example, treatment for prostate cancer can involve the temporary implantation of radioactive sources (e.g., rods) for a calculated period, followed by the subsequent removal of the radioactive sources. Alternatively, radioactive sources (e.g., seeds) can be permanently implanted in the patient and left to decay to an inert state over a predictable time. The use of temporary or permanent implantation depends on the isotope selected and the duration and intensity of treatment required.

Permanent implants for prostate treatment include radioisotopes with relatively short half lives and lower energies relative to temporary seeds. Exemplary permanently implantable sources include iodine-125, palladium-103 or cesium-131 as the radioisotope. The radioisotope can be encapsulated in a biocompatible casing (e.g., a titanium casing) to form a “seed” which is then implanted. Temporary implants for the treatment of prostate cancer may involve iridium-192 as the radioisotope. For temporary implants, radioactive rods are often used.

Conventional radioactive seeds are typically smooth sealed containers or capsules of a biocompatible material, e.g., titanium or stainless steel, containing a radioisotope within the sealed chamber that permits radiation to exit through the container/chamber walls. Other types of implantable radioactive sources for use in radiotherapy are radioactive rods and radioactive coils, as mentioned above.

Preferably, the implantation of radioactive sources for brachytherapy is carried out using minimally-invasive techniques such as, e.g., techniques involving needles and/or catheters. It is possible to calculate a desired location for each radioactive source which will give the desired radiation dose profile. This can be done using knowledge of the radioisotope content of each source, the dimensions of the source, accurate knowledge of the dimensions of the tissue or tissues in relation to which the source is to be placed, plus knowledge of the position of the tissue relative to a reference point. The dimensions of tissues and organs within the body for use in such dosage calculations can be obtained prior to or during placement of the radioactive sources by using conventional diagnostic imaging techniques including X-ray imaging, magnetic resonance imaging (MRI), computed tomography (CT) imaging, fluoroscopy and ultrasound imaging.

During the placement of the radioactive sources into position, a surgeon can monitor the position of tissues such as the prostate gland using, e.g., ultrasound imaging or fluoroscopy techniques which offer the advantage of low risk and convenience to both patient and surgeon. The surgeon can also monitor the position of the relatively large needle used in implantation procedures using ultrasound or other imaging.

A seed applicator, for example as shown in FIG. 1A and described below and in U.S. Pat. No. 5,860,909, can enable seeds to be implanted at fixed spaced-apart locations in a patient's body. Such applicators can include removable magazines that can be preloaded with seeds. Removable magazines of the prior art can supply a portion of a number of seeds required for a total treatment. It can be desirable in some circumstances to provide a substantially larger number of seeds in a single removable magazine than is currently provided.

### BRIEF DESCRIPTION OF THE DRAWINGS

FIG. 1A is a perspective view of an applicator in accordance with the prior art; FIG. 1B is a perspective view of a template for use with applicators of the prior art, and present invention.

FIG. 2A is a partial cross-sectional side view of an embodiment of an applicator in accordance with the present invention; FIG. 2B is a perspective view of the applicator of FIG. 2A; and FIG. 2C is a partial cross-sectional side view of an embodiment of a rotary clip for use with the applicator of FIG. 2A.

FIG. 3A is a perspective view of a clip for use in the applicator of FIG. 2A having a circle of ports; and FIG. 3B is a perspective view of an alternative clip for use in the applicator of FIG. 2A having two circles of ports.

FIG. 4A is a partial cross-sectional perspective view of the rotary clip and needle receiver of FIG. 3B; and FIG. 4B is a partial cross-sectional front view of the mechanism of FIGS. 3A and 3B, wherein the clip is unseated from the lower frame of the applicator.

FIG. 5A is a partial cross-sectional front view of an alternative embodiment of a needle receiver in accordance with the present invention; FIGS. 5B and 5C are partial cross-sectional front views of the mechanism of FIG. 5A wherein a rotary clip is seated within the needle receiver.

FIG. 6 is a perspective view of a still further embodiment of an applicator in accordance with the present invention having a magazine adapted to include strands.

### DETAILED DESCRIPTION

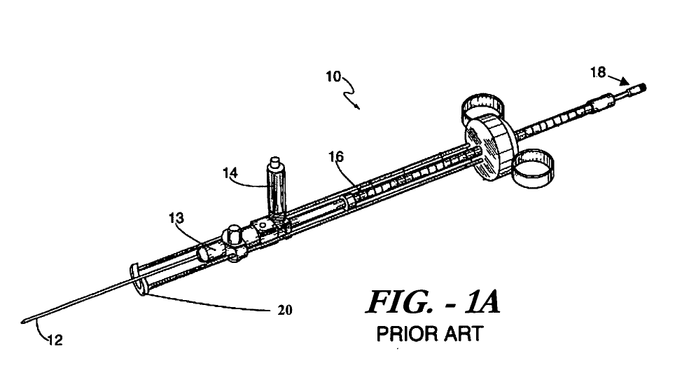

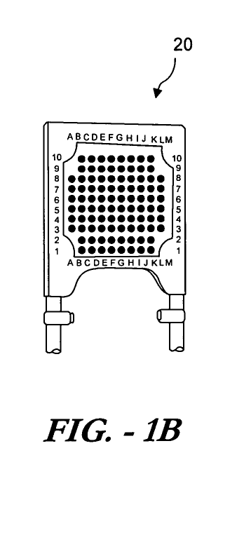

FIG. 1A illustrates a brachytherapy applicator 10 in accordance with the prior art. The applicator 10 includes a needle 12 insertable into a patient's body, a chuck 13 (also referred to herein as a needle receiver) for releasably holding the needle 12, a magazine 14 for holding and dispensing seeds into the needle receiver 13, a main barrel 16 connected to the needle receiver 13, and a stylet 18 extendable through the main barrel 16. The applicator 10 also includes a base frame member 20 configured to assist in positioning the applicator 10 relative to a template 20 (FIG. 1B). The magazine 14 extends away from the main barrel 16 and allows seeds to be fed to the main barrel 16 along the length of the magazine 14 in a fashion analogous to a Pez® dispenser.

Referring to FIG. 1B, a typical template 20 used to guide and/or inform the positioning of needles at the surgical site can provide access to more than one hundred locations. Although a physician need not necessarily employ needles at multiple sites, a typical procedure can require frequent replacements of relatively low-capacity seed magazines (e.g. 14) as commonly used in prior-art applicators (e.g. 10).

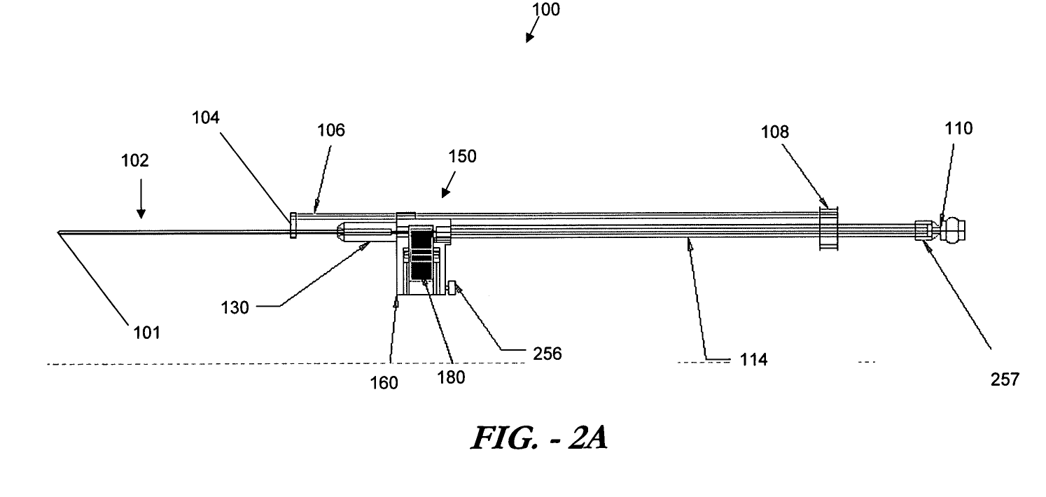

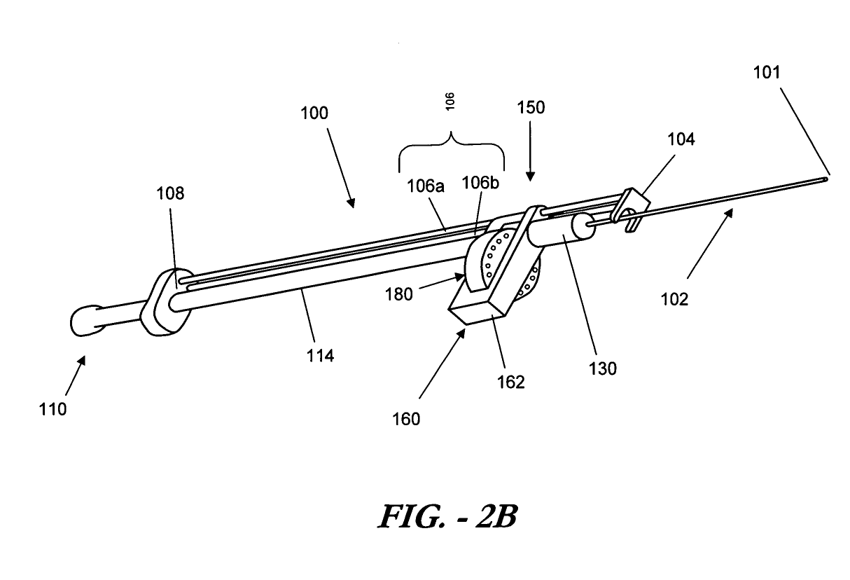

Referring to FIG. 2A, embodiments of an applicator 100 in accordance with the present invention can include a clip 180 employing rotary motion for providing to one or more implants (not shown) access to a needle 102. Such implants can include a radioactive source. The radioactive source can be a radioactive seed, a radioactive rod, or a radioactive coil, but is not limited thereto. The radioactive source can further be an anchor seed, which is a seed having an outer shape and/or outer coating adapted to resist movement once implanted at a desired location within the patient, for example, as disclosed in U.S. patent application Ser. No. 11/187,411, entitled “Implants for Use in Brachtherapy and Other Radiation Therapy That Resist Migration and Rotation,” filed Jul. 22, 2005, which is incorporated herein by reference. Alternatively, the implant can be some other object and need not be radioactive, e.g. the implant can be a spacer or a marker. The implant may also be a strand including spaced-apart radioactive sources. Embodiments of applicators 100 in accordance with the present invention can selectively accommodate any implant (or implants) meant to be inserted to a location by way of a needle 102, the implant being sized such that the implant can be accommodated by the needle 102 to which an applicator 100 can be removably connected. For reasons of convenience, embodiments will be described with reference to a “seed,” however it will be understood that embodiments can additionally or alternatively be used with any implant.

The applicator 100 of FIG. 2A can include a clip receiver 150 removably connected with a needle receiver 130 at a distal end and a push-rod housing 114 at a proximal end. The push-rod housing terminates near a push-rod retaining nut 257. The needle receiver 130 is sized to receive a needle 102, such as a MICK® needle or alternatively some other needle, such as a pre-load needle or a seed-lock needle. As shown, the distal end of the needle receiver 130 includes an exterior bevel, as well as a bore terminating in a funnel to ease insertion of a needle 102 within the needle receiver 130. The shape of the needle receiver 130 can be such as to be mate-able with multiple different needles, or alternatively the needle receiver 130 can be shaped and sized to receive a desired style of needle 102.

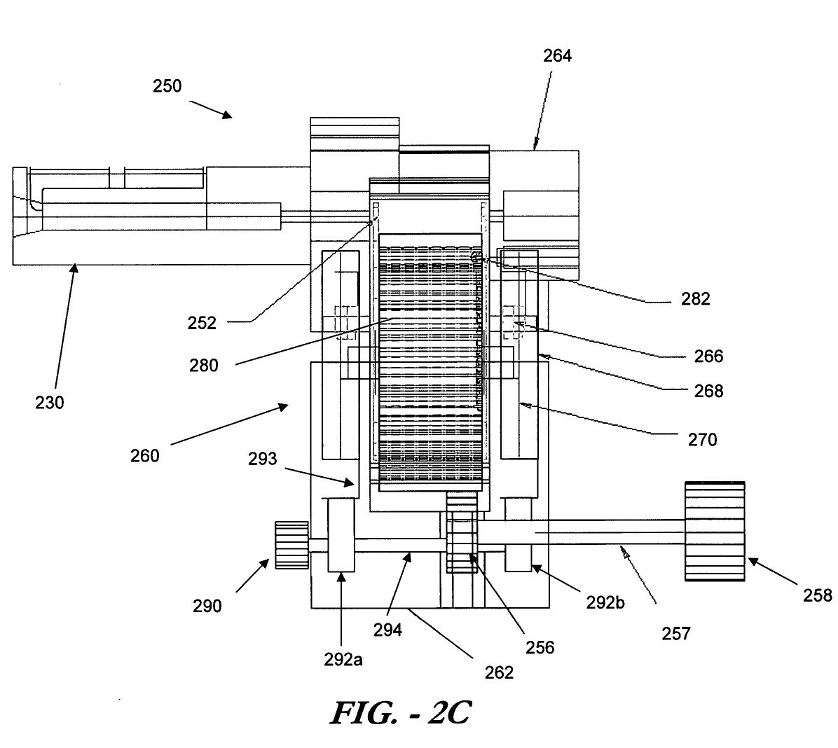

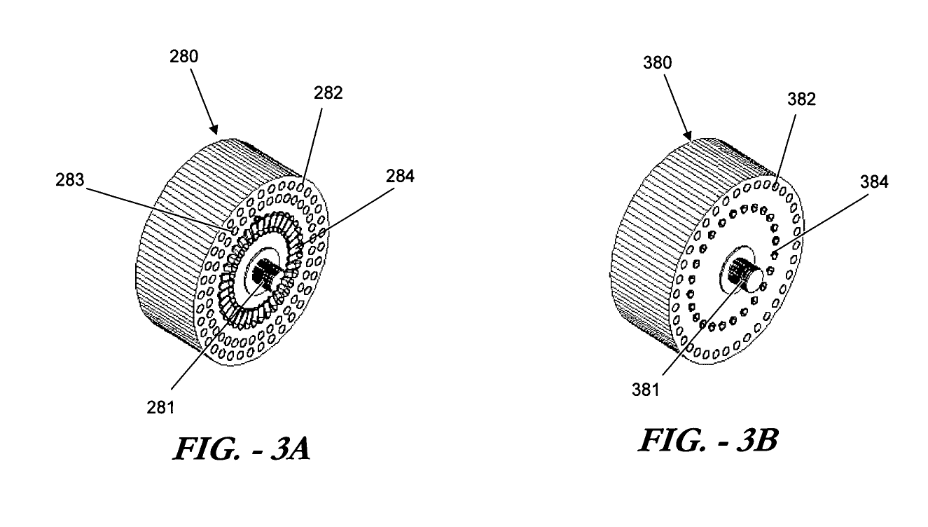

A channel can be formed between the push-rod housing 114 and the needle receiver 130 when a clip 180 positioned within the clip receiver 150 is arranged so that a port 282, 283 (as shown in FIG. 3A) of the clip 180 is aligned with the needle receiver 130 and the push-rod housing 114. The port 282, 283 bridges the needle receiver 130 and the push-rod housing 114 and when the channel is formed a push rod 110 (also referred to herein as a stylet) can be received through the channel. An implant, such as a seed, within the port 282, 283 can be expelled from the port and urged through the needle receiver 130 and into the needle 102. The implant can be further urged into position at the desired location, e.g. within a patient tissue. A distal end 101 of the needle 102 is typically inserted to the desired location within the surgical site before a proximal end of the needle 102 is mated with the needle receiver 130, thus the implant is typically urged to approximately the distal end 101 of the needle 102.

Referring to FIG. 2B, the push rod housing 114 can be provided with a guide frame 106 including one or more rods adjustably connected with the push rod housing 114. As shown, the guide frame 106 includes a pair of rods 106a, 106b that can optionally slide along the length of the applicator 100 through bores in the clip receiver 150. The rods 106a, 106b are movably connected with the push rod housing 114 by a seed depth selector handle 108. When the guide frame 106 is positioned as desired relative to the needle 102, the seed depth selector handle 108 can be selectably fixed to the push rod housing 114 to resist movement of the guide frame 106. The adjustability of the guide frame 106 allows the applicator 100 to be used with needles 102 intended to be arranged at different depths within the surgical site. The guide frame 106 as shown further includes a template guide 104 at a proximal end of the guide frame 106.

An embodiment of a clip receiver 250 (150 in FIGS. 2A and 2B) in accordance with the present invention is shown in FIG. 2C. The clip receiver 250 includes a rotary mechanism 260 for accomplishing rotary motion of the clip 280 (180 in FIGS. 2A and 2B). The clip receiver 250 can be selectively arranged in one or more positions to enable access to multiple circles of ports, thereby increasing a capacity of the clip 280. As shown in FIG. 3A, the clip 280 includes two circles of ports 282, 283, the circles of ports 282, 283 being accessible by repositioning the clip 280 relative to the push rod housing 114 and needle 102. Selective positioning of the clip 280 can be enabled by adjusting a position of a lower frame 262 of the rotary mechanism 260 relative to an upper frame 264. Myriad different mechanisms can be used to accomplish the selective positioning of the lower frame 262. For example, as shown in FIG. 2C, cams 292a, 292b connected by a shaft 294 and rotatable by way of a knob 290 can be employed to reposition the lower frame 262 by urging the cams 292a, 292b against a v-block 293 so that a spring force applied by a spring (not shown) to the lower frame 262 is overcome. The clip 280 is supported by the lower frame 262 and urged into position in the lower frame 262 by spring-loaded pins 266. Pins 270 can be employed to maintain alignment of the upper frame 264 and the lower frame 262. To urge the lower frame 262 toward the upper frame 264, and thereby urge a port 282, 283 into position so that a channel is formed, the cam 292a, 292b can be rotated so that the spring force can pull the lower frame 262 toward the upper frame 264 without resistance by the cams 292a, 292b. To enable positioning of multiple circles of ports 282, 283, the pins 270 and/or other mechanism components can have a location mark such as detents, grooves or slots (not shown) for holding the lower frame 262 in position. The lower frame 262 can be released and freed to move by a pin (not shown) that can be actuated by pushing, pulling, or pressing, etc.

In an alternative embodiment, the lower frame 262 can be designed to be adjusted manually by physically manipulating the lower frame 262 to find the location mark (e.g. detents, grooves or slots) along pins 270 of the mechanism. Alternatively some other mechanical device can be employed to enable the mechanism to reposition the clip 280 within the clip receiver 250 such that a port 282, 283 is accessible to the push rod housing 114 and needle 102. One of ordinary skill in the art after reading the above description will appreciate the myriad different mechanical devices and schemes by which movement of the lower frame 262 relative to the upper frame 294 can be accomplished, while still being within the scope of the present invention.

Once the clip 280 is in a desired position, such that a circle of ports 282, 283 is accessible to the push rod housing 114 and needle 102, the clip 280 can be rotated between ports 282, 283 arranged along a circle by way of a friction wheel 256 or gear. The friction wheel 256 can be rotated using a knob 258 connected with the friction wheel 256 by a shaft 257. As shown in FIGS. 2A and 2C, the knob 258 extends out from the lower frame 262 a small distance relative to the push rod housing 114. In an alternative embodiment, the shaft 257 can extend as desired. For example, the shaft 257 can extend the length of the push rod housing 114, with support and rigidity being provided by way of some other structure such as a seed depth selector handle 108 extending down the height of the clip receiver 260 and including a cavity through which the shaft 257 can pass. The present invention is not meant to be limited to mechanical devices as selectively chosen and described herein, but rather is meant to encompass all such mechanical devices as would be readily known to one of ordinary skill in the art in light of the teachings provided herein. Thus, where ease of access to the knob 258 is desired, the shaft 257 may be extended.

In an embodiment, the clip 280 can be rotated to allow access to any port 282,283 within the clip 280 at the desire of the physician. For example, in some embodiments, it may be desired that ports including spacers be grouped, while ports including radiation seeds are grouped separately. The relative ease of adjustment of the clip 280 allows the physician access to a desired implant.

Where a friction wheel 256 is employed, the friction wheel 256 (or at least its outer surface) can be formed of a semi-pliant material such as rubber. Alternatively, the friction wheel 256 can be formed of a rigid material such as plastic, or metal (e.g. aluminum, titanium, or surgical steel). Preferably, the peripheral surface area of the friction wheel 256 is textured or roughened so that when the friction wheel 256 rotates, the rotational motion is imparted to the clip 280 and the clip 280 is rotated to align an alternative port 282, 283 with the channel. The friction wheel 256 is rotatably connected with the lower frame 262 so that the friction wheel 256 remains in frictional contact with the clip 280 as the lower frame 262 is repositioned. In other embodiments a gear (not shown) can be employed to engage complementary mating structures. For example, the friction wheel 256 can include gear teeth that mesh with teeth on the outer surface or axel of the clip 280.

A position of the clip 280 relative to a port 282, 283 during movement of the clip 280 within the clip receiver 260 can be revealed by a spring-loaded pin (not shown) resting within one of multiple detent 284 of the clip 280, shown in FIG. 3A. When a port 282, 283 is aligned with the channel, the spring-loaded pin will rest within the detent 284. The user must overcome some spring force of the spring-loaded pin to rotate the clip 280 to another port 282, 283, thereby revealing alignment of a port 282, 283 to the user. As can be seen in FIG. 3A, the detents 284 can have a radial length accommodating movement of the spring-loaded pins during repositioning of the clip 280 from a circle of ports 282 to another circle of ports 283.

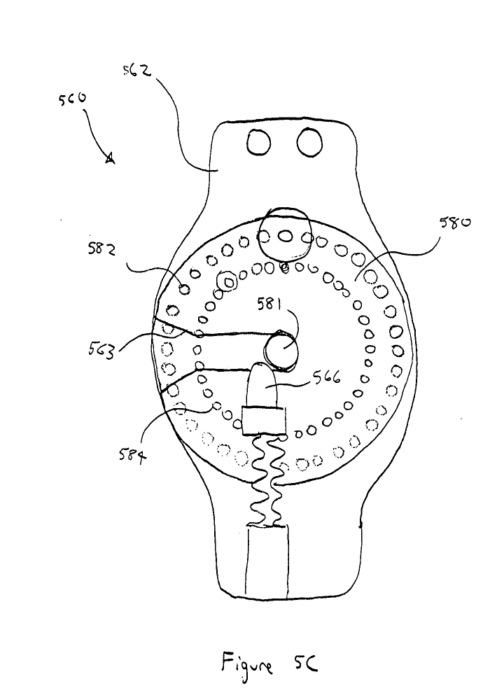

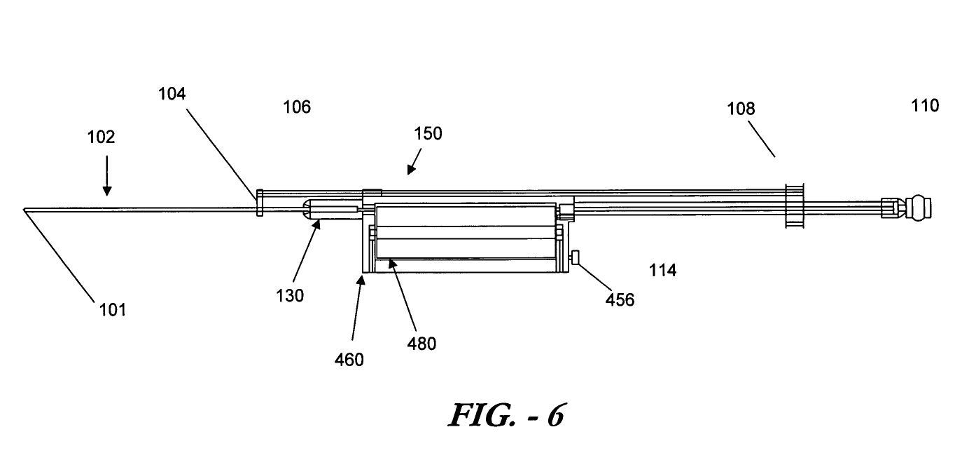

The clip 280 as shown includes ports 282, 283 spaced apart in circumferential increments of 10 degrees so that a total of 36 ports 282, 283 for each circle can be accessed. Thus, the clip 280 of FIG. 3A has an approximately 1 inch diameter and can accommodate as many as 72 implants. In other embodiments, clips for use with applicators of the present invention can be sized as desired and can include more or fewer ports spaced as can be accommodated. Further, the length of the clip along the push-rod housing can be varied to accommodate a desired implant. For example, as shown in FIG. 6 and described below, the clip length can be extended to accommodate anchor seeds, multiple seeds or strands.

As can be seen in FIG. 3B, embodiments of applicators in accordance with the present invention need not include a clip 280 having multiple circles of ports 282, 283, but rather can include a clip 380 having a single circle of ports 382. A clip 380 having a single circle of ports 382 can provide a higher implant capacity than a typical magazine 14 (as shown in FIG. 1A). The clip 380 includes ports 382 spaced apart in circumferential increments of 10 degrees so that a total of 36 ports 382 can be accessed. Therefore, the clip 380 can accommodate as many as 36 implants. As above, clips for use with applicators of the present invention can include more or fewer ports spaced as can be accommodated. In still other embodiments, more than two circles of ports can be employed. A number of circles of ports, and a number of ports within a circle are primarily defined by the diameter of the clip and the diameter of the port.

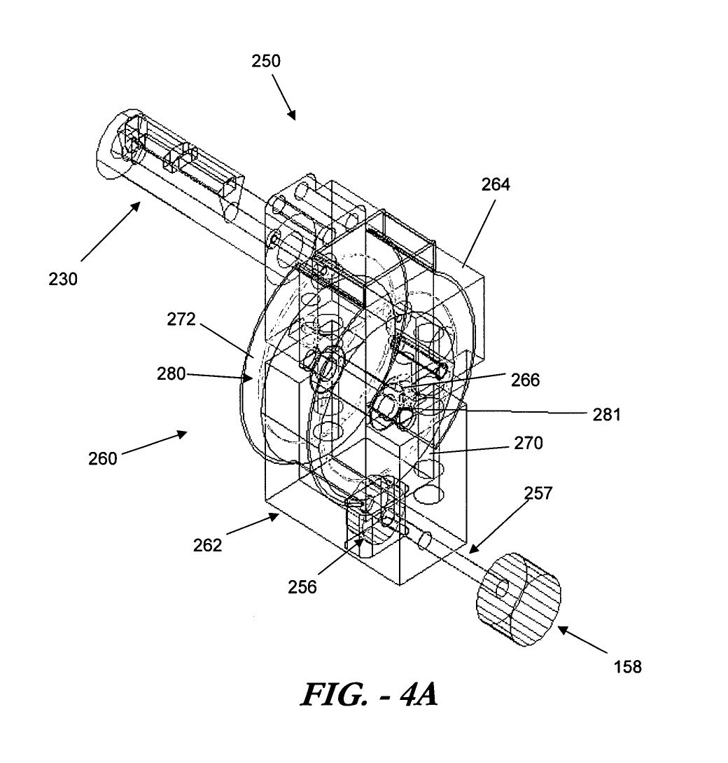

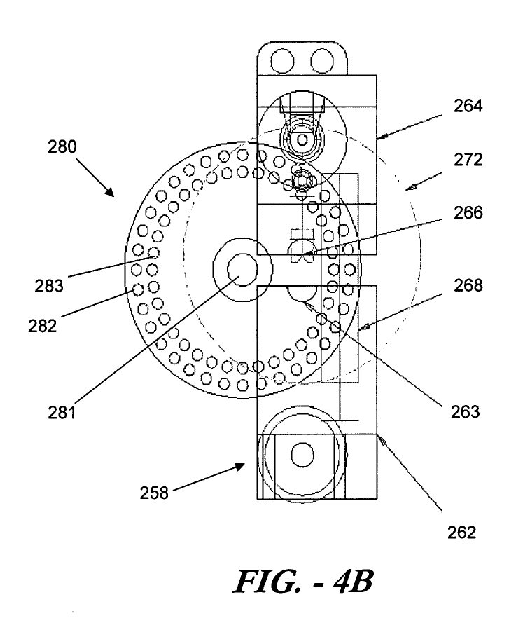

As can be seen in the perspective partial cross-sectional view of FIG. 4A, a shield 272 is positioned around the clip 280 to reduce or minify an amount of radiation that escapes from the applicator 100 where the implants placed in the ports are radioactive. The clip 280 can be formed using a transparent plastic, for example by molding. The clip 280 can further include a shaft 281 molded into the clip 280, or alternatively a shaft 281 made from a different material (e.g., aluminum, titanium or surgical steel) interference fit or otherwise fixedly connected with the clip 280. Where the clip 280 is formed of a plastic, the clip 280 does not sufficiently restrict radiation from escaping the clip 280; therefore, shielding is employed to prevent leakage. In other embodiments, clips for use in applicators of the present invention can be formed from a different material, such as aluminum, titanium or surgical steel. Further, in other embodiments clips for use in applicators in accordance with the present invention can be formed using a radiation blocking material. However, sources of radiation such as seeds are known to emit from their ends, where ports are unobstructed in order to bridge a push-rod housing and a needle receiver, therefore shielding can be required where radiation leakage is beyond an acceptable amount. The shielding can optionally include a window that is revealed when a portion of the shielding is repositioned (e.g., the portion can be a hinged door or a sliding piece). The window can be adapted to display an implant number by which a user can determine which port of multiple ports bridges the needle receiver and the push-rod housing.

FIG. 4B is a front view of the rotary mechanism 250 showing the clip 280 unseated from the lower frame 262. As can be seen, the lower frame 262 includes a groove 263 within which the shaft 281 of the clip 280 rests once loaded into the rotary mechanism 250. As can be seen, the shielding 272 has a shape roughly corresponding to the accessible portions of the clip 280 so that the ports 282, 283 are shielded once the clip 280 is seated in the lower frame 262. The shielding 272 is not continuous (i.e., has an opening), to provide access for at least placing a contents of a port 282, 283 into a channel with a push-rod. Additional shielding 272 block the periphery of the clip 282 from exposure. The additional shielding 272 can be fitted once the clip 280 is positioned within the clip receiver. (The sequence of assembly is unimportant, therefore shielding on either side of the applicator 100 can be fixed in place or connectable, so long as the clip 280 has access to the lower frame 262 and can thereby be loaded or unloaded.)

As can be seen, in operation the lower frame 262 can be urged away from the upper frame 264 and the clip 280 can be placed so that the shaft 281 is rotatably supported by the groove 263 of the lower frame 262. The spring-loaded pin 266 applies a force to the shaft 281 to assist in maintaining the shaft 281 rotatably positioned within the groove 263.

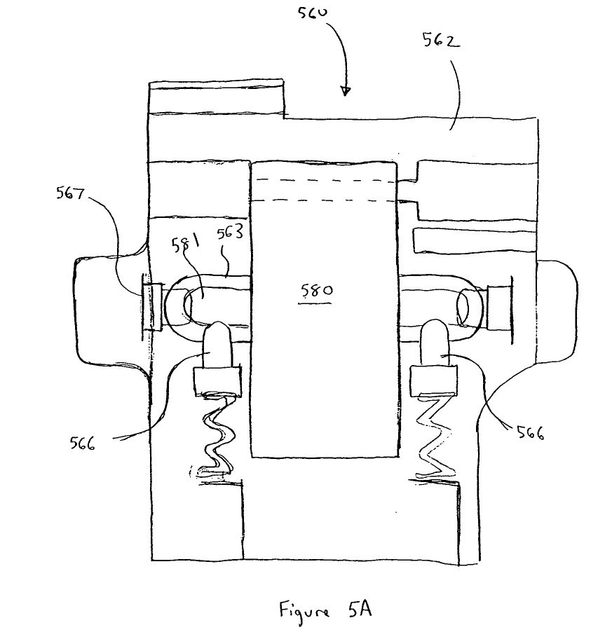

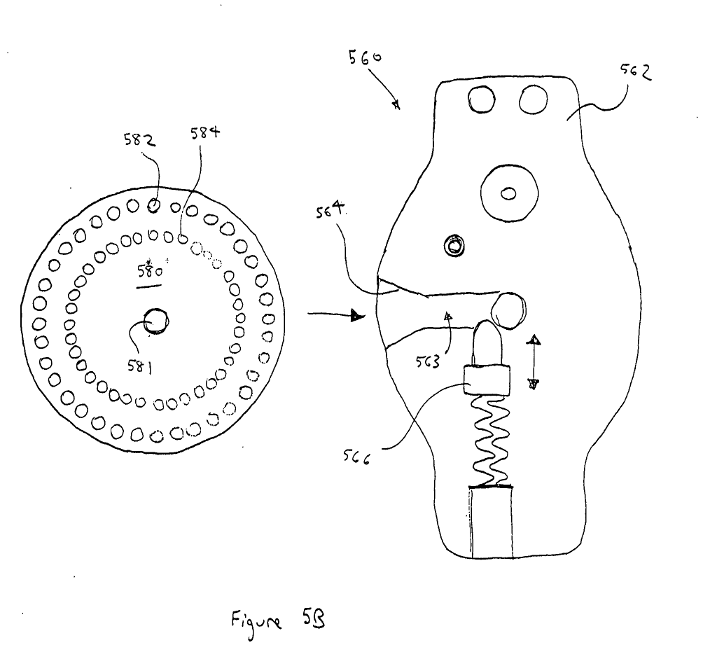

Referring to FIGS. 5A-5C, an alternative embodiment of a clip receiver 560 in accordance with the present invention is shown. The clip receiver 560 includes a frame 562 which accommodates a clip 580 without reconfiguration of the frame 562. The frame 562 as shown does not include a lower frame and an upper frame movable relative to one another. The frame 562 includes a loading track 563 for receiving the clip 580 and a clip retaining mechanism 566 to help hold the clip 580 in position. As shown, the clip retaining mechanism 566 comprises a pair of spring-loaded pins. The loading track 563 can include a funneled receiving end 564 to assist insertion of the clip 580 onto the loading track 563. The clip 580 is inserted into the funneled receiving end 564 and urged along the loading track 563 until a shaft 581 of the clip 580 contacts the spring-loaded pins 566. A force is applied to the clip 580 in the direction of insertion so that a spring force of the spring-loaded pins 566 is overcome and the spring-loaded pins 566 are depressed, allowing the clip 580 to be further urged along the loading track 563. In alternative embodiments, the clip retaining mechanism can comprise some other mechanism, such as a latch or insertable retaining pin, for example. One of ordinary skill in the art after reading the above description will appreciate the myriad different mechanisms that can be employed to retain a clip within the clip receiver.

As the clip 580 is further urged in a direction of insertion along the loading track 563, the clip 580 contacts a pair of horizontal position pins 567 arranged along the loading track 563 on each side of the clip 580. A force is applied to the clip 580 in the direction of insertion so that a spring force of the horizontal position pins 567 is overcome and the horizontal position pins 567 are depressed. The horizontal position pins 567 preferably include concave detents which are generally shaped to complement curved ends of the shaft 581. As the shaft 581 is received between the horizontal position pins 567, the shaft seats between the horizontal position pins 567. The shaft 581 is held between the horizontal position pins 567 and rotatable about an axis of the shaft 567. The spring-loaded pins 566 can return to an extended position obstructing the loading track 563 as the clip 580 is urged into place between the horizontal position pins 567, thereby providing resistance to movement of the clip 580 from a desired position within the clip receiver 560. The clip 580 is not restricted from rotating by the horizontal position pins 567, but to reposition the clip 580 along the loading track 563 a force must be applied to the clip 580 to overcome a retaining force applied by the horizontal position pins 567. Likewise, a spring force of the spring-loaded pins 566 must be overcome in order to remove the clip 580 from the clip receiver 560. However, overcoming the spring force is a relatively fast and easy process relative to an arrangement where the clip is sealed within a fixed carousel. Ease of removal allows a physician to replace a clip 580 with an alternative clip during a procedure or between procedures. In other embodiments, the clip receiver need not include horizontal position pins, or can include some other mechanism for rotatably holding a clip in place along the loading track.

While embodiments and variations thereof have been described with specificity in the above, the scope of the present invention is not intended to be limited to particular mechanisms named and described herein. In the mechanical arts, it is well known that different mechanisms can be employed to achieve similar movements, e.g., where a cam device has been described for positioning the lower frame relative to the upper frame, myriad different mechanical devices can be substituted for the cam device with varying degrees of success.

As mentioned above, embodiments of applicators in accordance with the present invention can include a clip and a clip receiver having appropriate lengths along the push-rod housing as required by the character of the implants. Referring to FIG. 6, there is shown a clip 480 and a clip receiver 460 sized to accommodate strands positioned within ports of the clip 480. Each strand can include a plurality of radioactive sources spaced apart from one another, e.g. in accordance with a treatment plan. The number of ports and length of the clip 480 potentially allow for an entire treatment plan to be provided in a single clip 480. In such scenarios, a clip 480 can be pre-loaded at a point of manufacture, and provided to the user without requiring the user to handle the materials contained therein. Such pre-loaded clips 480 offer benefits to hospitals or clinics that strive to minify the amount of handling of the implants performed by staff. It is also possible for a physician to load strands into the clip. As will be appreciated, and which can be extrapolated from the embodiments described, the clip and clip receiver can be longer or shorter as needed. It is also within the scope of the present invention that a port of the clip 480 includes an array of loose seeds and spacers axially arranged with respect to one another. For example, where an implant appropriate for a treatment plan is an anchor seed, the clip and clip receiver can have a length appropriate to the implant. As will be obvious to one of ordinary skill in the art in light of the above teachings, mechanisms as described above in FIGS. 2A-5B can be modified to accommodate clips and clip receivers having different shapes.

Embodiments of applicators of the present invention can be formed from myriad different materials. Where desired, the applicator can be disposable, and therefore can include components made of a polymer material, or the applicator can be reusable, and therefore can include components made of medical grade steel, or some similar acceptable material. Alternatively, the applicator can include a combination of disposable and reusable components. For example, in an embodiment the clip and the needle receiver can be disposable, and therefore can optionally comprise a polymer material.

The previous description of the preferred embodiments is provided to enable any person skilled in the art to make or use the embodiments of the present invention. While the invention has been particularly shown and described with reference to preferred embodiments thereof, it will be understood by those skilled in the art that various changes in form and details may be made therein without departing from the spirit and scope of the invention.

What is claimed is:

1. An applicator for positioning an implant at a surgical site, the applicator comprising:
a needle receiver adapted to be removably mateable with a needle positioned at the surgical site;
a housing adapted to receive a push rod; and
a clip having a plurality of ports adapted to bridge the needle receiver and the housing so that the needle receiver can receive the push rod, at least some of the plurality of ports accessible by a physician in a sequence desired by the physician;
wherein the clip is rotatably repositionable so that the plurality of ports can selectively bridge the needle receiver and the housing; and
wherein one or more implants are loadable into the ports of the clip.

2. The applicator of claim 1, wherein:
the needle receiver includes a bore and a bevel; and
the bevel is adapted to receive a hub of a first type of brachytherapy needle and the bore is adapted to receive a hub of a second type of brachytherapy needle.

3. The applicator of claim 1, wherein the clip is manually rotatable.

4. The applicator of claim 1, further comprising:
a clip receiver adapted to receive the clip; and
wherein the clip is received within the clip receiver to bridge the needle receiver and the housing so that the needle receiver can receive the push rod.

5. The applicator of claim 4, wherein the clip receiver includes a positioning device to arrange the clip in an initial position for receiving and removing the clip and a load position for bridging the needle receiver and the housing.

6. The applicator of claim 4, wherein the clip includes two or more circles of ports.

7. The applicator of claim 6, wherein the clip receiver includes a positioning device to arrange the clip in an initial position for receiving and removing the clip and two or more load positions for bridging the needle receiver and the housing, the two or more load positions corresponding to two or more circles of ports.

8. The applicator of claim 4, wherein:
the clip receiver includes a spring-loaded pin for providing resistance to rotation of the clip; and
the clip includes a plurality of detents for receiving the spring-loaded pin, the clip being rotatable by overcoming resistance provided by the spring-loaded pin.

9. A clip for use in retaining an implant for positioning at a surgical site, the clip comprising:
a shaft; and
two or more circles of ports disposed about the shaft for receiving the implant, each of the circles of ports comprising a plurality of ports.

10. The clip of claim 9, further comprising:
a plurality of detents for receiving a pin; and
wherein the two or more circles of ports are radially aligned with the plurality of detents so that the plurality of detents generally correspond to the plurality of ports.

11. The clip of claim 9, wherein a surface of the clip is textured to provide frictional resistance.

12. A system for positioning an implant at a surgical site, the system comprising:
a needle;
a push-rod;
an applicator including:
a needle receiver adapted to be removably mateable with the needle;
a housing adapted to receive the push rod; and
a clip receivable by the applicator, the clip having a plurality of ports adapted to bridge the needle receiver and the housing so that the needle receiver can receive the push rod;
wherein the clip is rotatably repositionable so that the plurality of ports can selectively bridge the needle receiver and the housing; and
wherein an implant is loadable into the ports of the clip.

13. The system of claim 12, wherein:
the needle is one of a first type of brachytherapy needle and a second type of brachytherapy needle;
the needle receiver includes a bore and a bevel; and
the bevel is adapted to receive a hub of the first type of brachytherapy needle and the bore is adapted to receive the hub of a second type of brachytherapy needle.

14. The system of claim 12, wherein the clip is manually rotatable.

15. The system of claim 12, wherein the applicator for comprises:
a clip receiver adapted to receive the clip; and
wherein the clip is received within the clip receiver to bridge the needle receiver and the housing so that the needle receiver can receive the push rod.

16. The system of claim 15, wherein the clip receiver includes a positioning device to arrange the clip in an initial position for receiving and removing the clip and a load position for bridging the needle receiver and the housing.

17. The system of claim 12, wherein the clip includes two or more circles of ports.

18. The system of claim 17, wherein the applicator for comprises:
a clip receiver adapted to receive the clip; and
wherein:
the clip is received within the clip receiver to bridge the needle receiver and the housing so that the needle receiver can receive the push rod.
the clip receiver includes a positioning device to arrange the clip in an initial position for receiving and removing the clip and two or more load positions for bridging the needle receiver and the housing, the two or more load positions corresponding to two or more circles of ports.

19. The system of claim 18, wherein:
the clip receiver includes a spring-loaded pin for providing resistance to rotation of the clip; and
the clip includes a plurality of detents for receiving the spring-loaded pin, the clip being rotatable by overcoming resistance provided by the spring-loaded pin.
